SEQUENCE LISTING (28 sequences)

SEQ ID NO: 1 - DNA, 18 bp, Artificial Sequence
  a-SMA forward primer
cactgccgcatcctcatc

SEQ ID NO: 2 - DNA, 21 bp, Artificial Sequence
  a-SMA reverse primer
tgctgttgtaggtggtttcat

SEQ ID NO: 3 - DNA, 24 bp, Artificial Sequence
  FSP1 forward primer
tgtcctgcatcgccatgatgtgta

SEQ ID NO: 4 - DNA, 24 bp, Artificial Sequence
  FSP1 reverse primer
tgaacttgctcagcatcaagcacg

SEQ ID NO: 5 - DNA, 21 bp, Artificial Sequence
  VEGF forward primer
aggaggagggcagaatcatca

SEQ ID NO: 6 - DNA, 21 bp, Artificial Sequence
  VEGF reverse primer
ctcgattggatggcagtagct

SEQ ID NO: 7 - DNA, 19 bp, Artificial Sequence
  MMP2 forward primer
gcggcggtcacagctactt

SEQ ID NO: 8 - DNA, 23 bp, Artificial Sequence
  MMP2 reverse primer
cacgctcttcagactttggttct

SEQ ID NO: 9 - DNA, 20 bp, Artificial Sequence
  OCT4 forward primer
ctgaagcagaagaggatcac

SEQ ID NO: 10 - DNA, 20 bp, Artificial Sequence
  OCT4 reverse primer
gaccacatccttctcgagcc

SEQ ID NO: 11 - DNA, 24 bp, Artificial Sequence
  SOX2 forward primer
aacgtttgccttaaacaagaccac

SEQ ID NO: 12 - DNA, 24 bp, Artificial Sequence
  SOX2 reverse primer
cgagataaacatggcaatcaaatg

SEQ ID NO: 13 - DNA, 23 bp, Artificial Sequence
  NANOG forward primer
cgaagaatagcaatggtgtgacg

SEQ ID NO: 14 - DNA, 21 bp, Artificial Sequence
  NANOG reverse primer
ttccaaagcagcctccaagtc

SEQ ID NO: 15 - DNA, 21 bp, Artificial Sequence
  GATA2 forward primer
gggctagggaacagatcgacg

SEQ ID NO: 16 - DNA, 20 bp, Artificial Sequence
  GATA2 reverse primer
gcagcagtcaggtgcggagg

SEQ ID NO: 17 - DNA, 22 bp, Artificial Sequence
  Brachy forward primer
atgagcctcgaatccacatagt

SEQ ID NO: 18 - DNA, 22 bp, Artificial Sequence
  Brachy reverse primer
tcctcgttctgataagcagtca

SEQ ID NO: 19 - DNA, 20 bp, Artificial Sequence
  C/EBPa forward primer
ctagagatctggctgtgggg

SEQ ID NO: 20 - DNA, 20 bp, Artificial Sequence
  C/EBPa reverse primer
tcataactccggtccctctg

SEQ ID NO: 21 - DNA, 20 bp, Artificial Sequence
  PU.1 forward primer
acggatctataccaacgcca

SEQ ID NO: 22 - DNA, 20 bp, Artificial Sequence
  PU.1 reverse primer
ggggtggaagtcccagtaat

SEQ ID NO: 23 - DNA, 22 bp, Artificial Sequence
  MIXL1 forward primer
agttggactgccttggtcactt

SEQ ID NO: 24 - DNA, 24 bp, Artificial Sequence
  MIXL1 reverse primer
acaaacctccgcctttcctctaca

SEQ ID NO: 25 - DNA, 19 bp, Artificial Sequence
  GATA1 forward primer
gggatcacactgagcttgc

SEQ ID NO: 26 - DNA, 19 bp, Artificial Sequence
  GATA1 reverse primer
acccctgattctggtgtgg

SEQ ID NO: 27 - DNA, 25 bp, Artificial Sequence
  GAPDH forward primer
gaaaycccatcaccaatcttccagg

SEQ ID NO: 28 - DNA, 22 bp, Artificial Sequence
  GAPDH reverse primer
gcaattgagccccagccttctc

(19) United States
(12) Patent Application Publication
(10) Pub. No.: US 2020/0248141 A1
(43) Pub. Date: Aug. 6, 2020

(54) COMPOSITION AND KIT FOR DIFFERENTIATING CANCER ASSOCIATED FIBROBLASTS INTO MACROPHAGES, AND METHOD OF USING THE SAME

(71) Applicant: KOREA INSTITUTE OF SCIENCE AND TECHNOLOGY, Seoul (KR)

(72) Inventors: So Yeon KIM, Seoul (KR); HyeRan GWAK, Seoul (KR); Eun Gyeong YANG, Seoul (KR)

(73) Assignee: KOREA INSTITUTE OF SCIENCE AND TECHNOLOGY, Seoul (KR)

(21) Appl. No.: 16/601,853

(22) Filed: Oct. 15, 2019

(30) Foreign Application Priority Data
Feb. 1, 2019 (KR) 10-2019-0013783

(51) Int. Cl. C12N 5/0786; A61K 35/15

(52) U.S. Cl. CPC C12N 5/0645; A61K 35/15; C12N 2506/1307; C12N 2501/2304; C12N 2501/115; C12N 2500/00; C12N 2500/30

(57) ABSTRACT

Provided are a composition and a kit for reprogramming cancer associated fibroblasts (CAFs) into macrophages, and a method of using the same. According to a method of reprogramming CAFs according to an aspect, macrophages may be prepared with a high yield in a short period of time, and the tumor microenvironment may be suppressed and macrophages produced by reprogramming CAF may elimininate cancer cells. Therefore, the macrophages may be usefully applied as an anticancer agent or an anticancer adjuvant.

### CROSS-REFERENCE TO RELATED APPLICATION

This application claims the benefit of Korean Patent Application No. 10-2019-0013783, filed on Feb. 1, 2019, in the Korean Intellectual Property Office, the disclosure of which is incorporated herein in its entirety by reference.

### REFERENCE TO SEQUENCE LISTING SUBMITTED VIA EFS-WEB

This application includes an electronically submitted sequence listing in .txt format. The .txt file contains a sequence listing entitled “1183-0138PUS1 ST25.txt” created on Dec. 9, 2019 and is 5,803 bytes in size. The sequence listing contained in this .txt file is part of the specification and is hereby incorporated by reference herein in its entirety.

### BACKGROUND

### 1. Field

The present disclosure relates to a composition and a kit for differentiating cancer-associated fibroblasts into macrophages, and a method of using the same.

### 2. Description of Related Art

Recently, cancer immunotherapy has been extensively advancing and emerged as an important treatment modality in clinical practice. However, the treatment outcomes thereof are poor at only around 20% of treated patients. One of the reasons for this limitation is that tumor tissue is usually enclosed by extracellular matrix (ECM), and their microenvironment is not supportive for immune system. Therefore, there is a demand for a more advanced and diversified form of cancer-specific immunotherapeutic technique capable of overcoming the tumor microenvironment and expanding cancer immunotherapeutic effects.

The tumor microenvironment consists of various components including a thick and dense ECM that surrounds a tumor, cancer supporting cells that assist the growth of cancer cells, cancer suppressive cells that suppress or extinguish the growth of cancer cells. Among them, major cells that assist the growth and proliferation of cancer cells include cancer-associated fibroblasts (CAFs), tumor-associated macrophages (TAMs). In particular, CAFs prevent penetration of a drug or an immunotherapeutic agent into tumor tissue by creating the thick and dense ECM. In addition, CAFs secrete a variety of cytokines known to assist blood vessel formation to support both nutrient and oxygen for tumor growth and contribute to drug resistance and immune suppression. The origin of CAF has been elucidated as normal stromal cells (fibroblasts, vascular cells, mesenchymal stem cells, and adipocytes) and these normal cells become activated by various cytokines secreted from cancer cells. Due to these cancer supportive roles, it is important to make strategies to eliminate cancer cells as well as CAF and other cancer supportive cells.

As such, we hypothesized that tumor supportive microenvironment can be shifted to tumor suppressive environment when CAFs are reprogrammed and differentiated into macrophages, which can actively eliminate cancer cells as well. Furthermore, accessibility of other drugs and immune cells can be enhanced due to the destruction of tumor microenvironment enclosed ECM and CAF.

### SUMMARY

An aspect provides a method of reprogramming cancer-associated fibroblasts (CAFs) into macrophages, a method of enhancing expression of Oct4 and Sox2 in CAFs and culturing the CAFs in a medium to differentiate the CAFs into induced pluripotent stem cells (iPCs); culturing the iPCs in a medium to differentiate the iPCs into hematopoietic stem cells; and culturing the hematopoietic stem cells in a medium to differentiate the hematopoietic stem cells into macrophages.

Additional aspects will be set forth in part in the description which follows and, in part, will be apparent from the description, or may be learned by practice of the presented embodiments of the disclosure.

According to the method described in [005], a cancer supporting cell, CAF is reprogrammed to be a macrophage, cancer suppressive cells. When CAFs are reprogrammed, ECM production, promotion of blood vessel formation, and cancer growth and metastasis by CAFs may be prevented, and macrophages may directly kill surrounding cancer cells, and therefore, the tumor microenvironment may be changed. Further, destruction of the tumor microenvironment may enhance the penetration of existing immune cells or other immunotherapeutics, and thus it may be applied as an anticancer adjuvant to further enhance effects of previously developed anti-cancer immunotherapeutics.

As used herein, the term “fibroblast” may refer to a cell of the connective tissue of a mammal, which constitutes components of the fibrous connective tissue. As used herein, the term “cancer-associated fibroblast (CAF)” refers to a kind of cells existing in the tumor stroma, and is known to be involved in the tumor growth, formation of tumor blood vessels, and invasion and metastasis of tumor cells in most cancers.

The CAF may be isolated from a sample of a cancer tissue. As used herein, the term “cell isolation” may mean removal of at least 20%, 30%, 40%, 50%, 60%, 70%, 80%, 90%, 95%, or 99% of cells, which are normally connected with cells to be isolated, from a non-treated tissue. With regard to a cell population including cells obtained from a tissue, when other cells normally connected with the cells in the tissue in a non-treated state are less than 50% of the entire cells, it may mean that the cells are “isolated”. As used herein, “isolated” may mean a naturally occurring tissue, or a tissue or a cell existing in an environment different from an intracellular environment. For example, when cells naturally occur in a multicellular organ and the cells are removed from the multicellular organ, the cells are those “isolated”.

In a specific embodiment, the CAF may have increased expression of CAF markers, for example, α-SMA, FSP-1, VEGF, and/or MMP2, as compared with normal fibroblasts.

In the method, the enhancing of expression of Oct4 and Sox2 in CAFs and culturing of the CAFs in a medium to differentiate the CAFs into iPCs will be described in detail as follows.

As used herein, the term “enhancing of expression of octamer binding transcription factor 4 (Oct4) and SRY-box2 (Sox2)” may refer to a known method that increases the expression of Oct4 and Sox2 genes or proteins in CAFs, as compared with control CAFs. The control CAFs may be CAFs before enhancing expression of the genes or proteins, for example, wild-type CAFs. The enhancing of expression may be performed by, for example, transduction via a viral vector, protein injection, treatment of a chemical cocktail which is known to express Oct4 or Sox2, or treatment of miRNA capable of increasing expression of Oct4 and/or Sox2. In a specific embodiment, the enhancing of expression may be achieved by transducing Oct4 and Sox2 genes into CAFs. The transducing may be achieved by a known method, for example, using a lentiviral vector, but is not limited thereto.

In the method, CAFs in which Oct4 and Sox2 expression are enhanced may be cultured in a medium to be differentiated into iPCs. Further, in the method, miR125b expression may be further enhanced.

As used herein, the term “induced pluripotent stem cells (iPCs)” refer to cells that are able to develop into a complete organism. Pluripotent cells include cells at an early embryonic stage, together with fertilized egg cells. Pluripotent cells refer to exponentially divided embryonic stem cells derived from the internal cell mass of blastocysts, which are able to generally differentiate into all three germ cells of mesoderm, endoderm, and ectoderm. As used herein, the term “induced pluripotent stem cell (iPSC)” may refer to a pluripotent stem cell which is prepared by artificially inducing expression of a particular gene in a non-pluripotent adult somatic cell.

In the method, the CAFs may be cultured in a medium including a serum replacement, β-mercaptoethanol, a basic fibroblast growth factor (bFGF), or a combination thereof. The serum replacement may be a knockout serum replacement.

A concentration of the serum replacement may be about 1% by weight to about 20% by weight, about 2% by weight to about 18% by weight, about 5% by weight to about 15% by weight, about 6% by weight to about 14% by weight, about 7% by weight to about 13% by weight, or about 8% by weight to about 12% by weight of the total medium. A concentration of the β-mercaptoethanol may be about 0.05 mM to about 1.5 mM, about 0.06 mM to about 1.4 mM, about 0.07 mM to 1.3 mM, about 0.08 mM to 1.2 mM, or about 0.09 mM to 1.1 mM. A concentration of the bFGF may be about 1 ng/ml to about 20 ng/ml, about 2 ng/ml to about 18 ng/ml, about 5 ng/ml to about 15 ng/ml, about 6 ng/ml to about 14 ng/ml, about 7 ng/ml to about 13 ng/ml, or about 8 ng/ml to about 12 ng/ml.

The medium may further include glutamine, non-essential amino acids, penicillin, ora combination thereof.

The medium may be one or more selected from the group consisting of Dulbecco's Modified Eagle's Medium (DMEM), Minimal Essential Medium (MEM), Basal Medium Eagle (BME), RPMI 1640, DMEM/F-10 (Dulbecco's Modified Eagle's Medium: Nutrient Mixture F-10), DMEM/F-12 (Dulbecco's Modified Eagle's Medium: Nutrient Mixture F-12), α-Minimal essential Medium (α-MEM), Glasgow's Minimal Essential Medium (G-MEM), Isocove's Modified Dulbecco's Medium (IMDM), and KnockOut DMEM. The medium may be a DMEM/F-12 medium.

The cells may be grown in adherent cell culture. During the adherent cell culture, the cells may be adherent on a geltrex-coated cell support, for example, plate.

The culturing may be continued for about 10 days to about 20 days, about 11 days to 19 days, about 12 days to 18 days, about 13 days to 17 days, or about 14 days to 16 days.

In a specific embodiment, when expression of Oct4 and Sox2 may be enhanced or expression of miR125b may be optionally enhanced in the CAFs, which may be then cultured in the medium, CAFs may acquire pluripotency, thereby differentiating into iPSCs. The iPSC may be a cell expressing Oct4, Sox2, and Nanog which are pluripotency markers.

In the method, the culturing of the iPSCs in a medium to differentiate the iPSCs into hematopoietic stem cells will be described in detail as follows.

As used herein, the term “hematopoietic stem cell” may refer to a cell that produces blood cells such as leukocytes, erythrocytes, platelets, etc. through self-replication and differentiation in the bone marrow.

In the method, the iPSCs may be cultured in a medium including β-mercaptoethanol, fetal calf serum (FCS), or a combination thereof.

A concentration of the β-mercaptoethanol may be about 0.05 mM to about 1.5 mM, about 0.06 mM to about 1.4 mM, about 0.07 mM to about 1.3 mM, about 0.08 mM to about 1.2 mM, or about 0.09 mM to about 1.1 mM. A concentration of the FCS may be about 10% by weight to about 30% by weight, about 12% by weight to about 28% by weight, about 15% by weight to about 25% by weight, about 17% by weight to about 23% by weight, or about 16% by weight to about 22% by weight of the total medium.

The medium may further include non-essential amino acids, penicillin, streptomycin, or a combination thereof.

The medium may be one or more selected from the group consisting of Dulbecco's Modified Eagle's Medium (DMEM), Minimal Essential Medium (MEM), Basal Medium Eagle (BME), RPMI 1640, DMEM/F-10 (Dulbecco's Modified Eagle's Medium: Nutrient Mixture F-10), DMEM/F-12 (Dulbecco's Modified Eagle's Medium: Nutrient Mixture F-12), α-Minimal essential Medium (α-MEM), Glasgow's Minimal Essential Medium (G-MEM), Isocove's Modified Dulbecco's Medium (IMDM), and KnockOut DMEM. The medium may be a KnockOut DMEM.

The culturing may be non-adherent culturing. By the non-adherent culturing, cells may be cultured while forming a spherical shape.

The culturing may be continued for about 10 days to about 20 days, about 11 days to 19 days, about 12 days to 18 days, about 13 days to 17 days, or about 14 days to 16 days.

In a specific embodiment, when iPSCs are cultured in the medium, iPSCs may acquire ability to differentiate into hematopoietic stem cells, thereby differentiating into hematopoietic stem cells. The hematopoietic stem cells may be those expressing CD34 protein. A method of identifying the expression of CD34 protein may be performed by flow cytometry which is a known method.

In a specific embodiment, when iPSCs derived from CAFs, in which Sox2 and Oct4 are enhanced, or optionally, miR125b is enhanced, are differentiated into hematopoietic stem cells by the method according to one aspect, the highest expression of CD34 and CD45 proteins is observed.

Further, in a specific embodiment, when iPSCs derived from CAFs, in which Sox2 and Oct4 are enhanced, or optionally, miR125b is enhanced, are differentiated into hematopoietic stem cells by the method according to one aspect, the highest expression of GATA2 and/or Brachy which are mesodermal lineage markers is observed.

Further, in a specific embodiment, when iPSCs derived from CAFs, in which Sox2 and Oct4 are enhanced, or optionally, miR125b is enhanced, are differentiated into hematopoietic stem cells by the method according to one aspect, excellent embroinic body (EB)-formation ability is observed.

The hematopoietic stem cells obtained by the method according to an aspect have ability to differentiate into macrophages.

In the method, the culturing of the hematopoietic stem cells in a medium to differentiate the hematopoietic stem cells into macrophages will be described in detail as follows.

As used herein, the term “macrophage” may refer to a cell responsible for immunity, as a kind of cancer suppressive cells.

In the method, the hematopoietic stem cells may be cultured in a medium including IL-4, M-CFS, or a combination thereof.

A concentration of the IL-4 may be about 1 μg/ml to about 20 μg/ml, about 2 μg/ml to about 18 μg/ml, about 5 μg/ml to about 15 μg/ml, about 6 μg/ml to about 14 μg/ml, about 7 μg/ml to about 13 μg/ml, or about 8 μg/ml to about 12 μg/ml. A concentration of the M-CFS may be about 1 μg/ml to about 20 μg/ml, about 2 μg/ml to about 18 μg/ml, about 5 μg/ml to about 15 μg/ml, about 6 μg/ml to about 14 μg/ml, about 7 μg/ml to about 13 μg/ml, or about 8 μg/ml to about 12 μg/ml of the total medium.

The medium may further include FBS.

The medium may be one or more selected from the group consisting of Dulbecco's Modified Eagle's Medium (DMEM), Minimal Essential Medium (MEM), Basal Medium Eagle (BME), RPMI 1640, DMEM/F-10 (Dulbecco's Modified Eagle's Medium: Nutrient Mixture F-10), DMEM/F-12 (Dulbecco's Modified Eagle's Medium: Nutrient Mixture F-12), α-Minimal essential Medium (α-MEM), Glasgow's Minimal Essential Medium (G-MEM), Isocove's Modified Dulbecco's Medium (IMDM), and KnockOut DMEM. The medium may be an RPMI 1640 medium.

The culturing may be adherent-culturing.

The culturing may be continued for about 5 days to about 10 days, about 6 days to about 9 days, or about 7 days to about 8 days.

In a specific embodiment, when the hematopoietic stem cells are cultured in the medium, the hematopoietic stem cells may acquire functionality of macrophages, thereby differentiating into macrophages. The macrophage may exert a phagocytic function. The macrophage may exhibit an effect of preventing or treating cancer.

In a specific embodiment, the macrophages differentiated by the method according to an aspect may have increased expression of C/EBPα, PU.1, MXIL1, and/or GATA1, as compared with macrophages differentiated by other methods.

Another aspect provides macrophages prepared by the above method.

The method is the same as described above.

Since the macrophages may be prepared from CAFs, an effect of removing tumor-associated cells from the tumor microenvironment may be obtained. The tumor microenviroment is known to affect the tumor growth, tumor blood vessel formation, and invasion and metastasis of tumor cells, such as increased malignancy of cancer cells by promoting interactions in the tumor microenvironment and by increasing invasion of cancer cells. Therefore, since the macrophages may be used to change the tumor microenvironment, they may be applied as an adjuvant of anticancer agents or anticancer immunotherapeutic agents.

Further, the macrophage may exhibit an effect of directly killing and suppressing cancer cells, and thus it may be applied as an anticancer agent.

Still another aspect provides a pharmaceutical composition for preventing or treating cancer, the pharmaceutical composition including the macrophage prepared by the above method.

Still another aspect provides use of the macrophages prepared by the above method as a cell therapeutic agent for preventing or treating cancer.

The pharmaceutical composition may further include a physiologically acceptable matrix or a physiologically acceptable excipient, in addition to the cell. A type of the matrix and/or the excipient may depend on others according to an intended route of administration. Optionally, the pharmaceutical composition may further include other suitable excipients or active ingredients which are used together in the stem cell therapy.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 shows a graph of expression levels of α-SMA, FSP-1, VEGF, and MMP2, which are CAF markers, in cancer-associated fibroblasts, confirmed by qRT-PCR;

FIG. 2A shows results of observing iPCs under a fluorescence microscope, the iPCs obtained by transducing Oct4, Sox2, Oct4/Sox2, miR125b, or Sox2/miR125b into CAFs;

FIG. 2B shows results of observing iPCs under a fluorescence microscope, the iPCs obtained by transducing Oct4, Sox2, Oct4/Sox2, miR125b, or Sox2/miR125b into normal fibroblasts;

FIG. 3A shows qRT-PCR results of analyzing the expression of Oct4, Sox2, and Nanog, which are pluripotency markers, in iPCs, the iPCs being obtained by transducing Oct4, Sox2, Oct4/Sox2, miR125b, or Sox2/miR125b into CAFs;

FIG. 3B shows qRT-PCR results of analyzing the expression of Oct4, Sox2, and Nanog, which are pluripotency markers, in iPCs, the iPCs being obtained by transducing Oct4, Sox2, Oct4/Sox2, miR125b, or Sox2/miR125b into normal fibroblasts;

FIG. 4 shows results of analyzing whether iPCs obtained from CAFs by a method according to an aspect have EB-forming ability;

FIG. 5A shows flow cytometry results of analyzing the expression of CD34 which is a membrane protein of hematopoietic stem cells differentiated from iPCs which were obtained by transducing Oct4, Sox2, Oct4/Sox2, miR125b, or Sox2/miR125b into CAFs;

FIG. 5B shows flow cytometry results of analyzing the expression of CD34 which is a membrane protein of hematopoietic stem cells differentiated from iPCs which were obtained by transducing Oct4, Sox2, Oct4/Sox2, miR125b, or Sox2/miR125b into normal fibroblasts;

FIG. 6A shows qRT-PCR results of analyzing the expression of GATA2 and Brachy in hematopoietic stem cells differentiated from the iPCs which were obtained by transducing Oct4, Sox2, Oct4/Sox2, miR125b, or Sox2/miR125b into CAFs;

FIG. 6B shows qRT-PCR results of analyzing the expression of GATA2 and Brachy in hematopoietic stem cells differentiated from the induced pluripotent stem cells which were obtained by transducing Oct4, Sox2, Oct4/Sox2, miR125b, or Sox2/miR125b into normal fibroblasts;

FIG. 7A shows flow cytometry results of analyzing the expression of CD45 which is a cell membrane marker for blood cells/monocytes in hematopoietic stem cells which were obtained by transducing Oct4, Sox2, Oct4/Sox2, miR125b, or Sox2/miR125b into CAFs;

FIG. 7B shows flow cytometry results of analyzing the expression of CD45 which is a cell membrane marker for blood cells/monocytes in hematopoietic stem cells which were obtained by transducing Oct4, Sox2, Oct4/Sox2, miR125b, or Sox2/miR125b into normal fibroblasts;

FIG. 8A shows qRT-PCR results of analyzing the expression of C/EBPα, PU.1, MXIL1, and GATA1 in macrophages which were obtained by transducing Oct4, Sox2, Oct4/Sox2, miR125b, or Sox2/miR125b into CAFs.

FIG. 8B shows qRT-PCR results of analyzing the expression of C/EBPα, PU.1, MXIL1, and GATA1 in macrophages which were obtained by transducing Oct4, Sox2, Oct4/Sox2, miR125b, or Sox2/miR125b into normal fibroblasts;

FIG. 9A shows results of evaluating phagocytic function of macrophages which were obtained by transducing Oct4, Sox2, Oct4/Sox2, miR125b, or Sox2/miR125b into CAFs;

FIG. 9B shows results of evaluating phagocytic function of macrophages which were obtained by transducing Oct4, Sox2, Oct4/Sox2, miR125b, or Sox2/miR125b into normal fibroblasts; and

FIG. 10 is an illustration showing the preparation of macrophages from cancer-associated fibroblasts.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

### EXAMPLE 1

Preparation of Macrophages from Cancer-Associated Fibroblasts

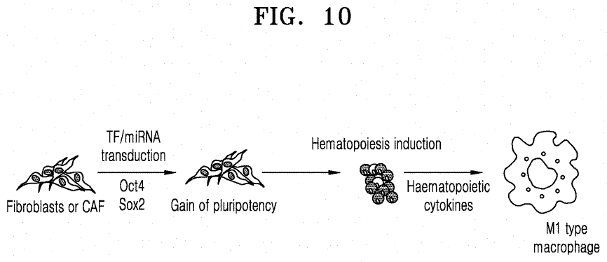

To prepare macrophages from CAFs, differentiation factors were first transduced into CAFs to prepare induced pluripotent stem cells, which were then differentiated into hematopoietic stem cells, and from the hematopoietic stem cells, macrophages were differentiated. An illustration showing the preparation of macrophages from CAFs is as shown in FIG. 10.

1.1. Preparation of Cell Line and Vector

CAFs (CAF, Catalog # CAF05) derived from human colon cancer were purchased from Neuromics (USA). As a control group, MRC5 which is a normal lung fibroblast was purchased from ATCC (USA) and used.

The differentiation factors to be transduced into the fibroblasts were Oct4, Sox2, and/or miR125b, and lentiviral vectors (pLM-mCitrin-Sox2, pLM-vexGFP-Oct4, and pMG-MIR-125b-1) for their expression were purchased from Addgene, and LPP-mCherry-HmiR125b was prepared in Macrogen (Korea).

To characterize the used fibroblasts, expression patterns of α-SMA, FSP-1, VEGF, and MMP2P which are reported as CAF markers were examined by qRT-PCR. RNAs in the cells were isolated using Trizol, and cDNA was synthesized using 1 μg of RNA, and primers of Table 1 were used to perform qRT-PCR. The relative expression level of the CAF marker in the CAFs was shown by taking the amount of the CAF marker in the normal fibroblasts as 1.

TABLE 1

| Name | | | |
|---|---|---|---|
| of | | | |
| gene | 5′-> 3′ | sequence | Note |
| a-SMA | Forward | CACTGCCGCATCCTCATC | SEQ ID NO: 1 |
| | Reverse | TGCTGTTGTAGGTGGTTTCAT | SEQ ID NO: 2 |
| FSP1 | Forward | TGTCCTGCATCGCCATGATGT | SEQ ID NO: 3 |
| | | GTA | |
| | Reverse | TGAACTTGCTCAGCATCAAGCACG | SEQ ID NO: 4 |
| VEGF | Forward | AGGAGGAGGGCAGAATCATCA | SEQ ID NO: 5 |
| | Reverse | CTCGATTGGATGGCAGTAGCT | SEQ ID NO: 6 |
| MMP2 | Forward | GCGGCGGTCACAGCTACTT | SEQ ID NO: 7 |
| | Reverse | CACGCTCTTCAGACTTTGGTTCT | SEQ ID NO: 8 |
| OCT4 | Forward | CTGAAGCAGAAGAGGATCAC | SEQ ID NO: 9 |
| | Reverse | GACCACATCCTTCTCGAGCC | SEQ ID NO: 10 |
| SOX2 | Forward | AACGTTTGCCTTAAACAAGACCAC | SEQ ID NO: 11 |
| | Reverse | CGAGATAAACATGGCAATCAAATG | SEQ ID NO: 12 |
| NANOG | Forward | CGAAGAATAGCAATGGTGTGACG | SEQ ID NO: 13 |
| | Reverse | TTCCAAAGCAGCCTCCAAGTC | SEQ ID NO: 14 |
| GATA2 | Forward | GGGCTAGGGAACAGATCGACG | SEQ ID NO: 15 |
| | Reverse | GCAGCAGTCAGGTGCGGAGG | SEQ ID NO: 16 |
| Brachy | Forward | ATGAGCCTCGAATCCACATAGT | SEQ ID NO: 17 |
| | Reverse | TCCTCGTTCTGATAAGCAGTCA | SEQ ID NO: 18 |
| C/EBPa | Forward | CTAGAGATCTGGCTGTGGGG | SEQ ID NO: 19 |
| | Reverse | TCATAACTCCGGTCCCTCTG | SEQ ID NO: 20 |
| PU.1 | Forward | ACGGATCTATACCAACGCCA | SEQ ID NO: 21 |
| | Reverse | GGGGTGGAAGTCCCAGTAAT | SEQ ID NO: 22 |
| MIXL1 | Forward | AGTTGGACTGCCTTGGTCACTT | SEQ ID NO: 23 |
| | Reverse | ACAAACCTCCGCCTTTCCTCTA | SEQ ID NO: 24 |
| | | CA | |
| GATA1 | Forward | GGGATCACACTGAGCTTGC | SEQ ID NO: 25 |
| | Reverse | ACCCCTGATTCTGGTGTGG | SEQ ID NO: 26 |
| GAPDH | Forward | GAAAYCCCATCACCAATCTTCC | SEQ ID NO: 27 |
| | | AGG | |
| | Reverse | GCAATTGAGCCCCAGCCTTCTC | SEQ ID NO: 28 |

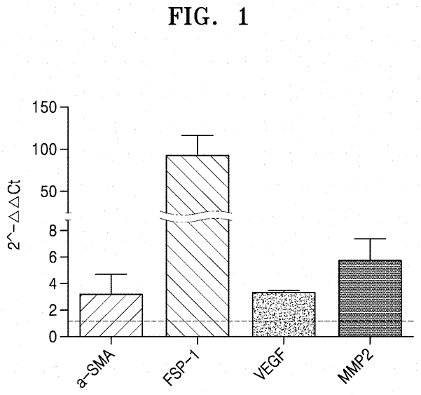

FIG. 1 shows a graph of expression levels of α-SMA, FSP-1, VEGF, and MMP2, which are CAF markers, in CAFs, confirmed by qRT-PCR.

As shown in FIG. 1 CAF markers were overexpressed in the used CAFs, as compared with normal fibroblasts.

1.2. Preparation of iPCs from CAFs

Lentiviral vectors for expression of Oct4, Sox2, and miR125b genes were transduced into CAFs to prepare iPCs, respectively. Specifically, packaging DNA (TAKARA) used for formation of viral particles, and each viral vector of pLM-mCitrin-Sox2, pLM-vexGFP-Oct4, and LPP-mCherry-HmiR125b were combined at a ratio of 3:1, and then transduced into a host cell HEK293T, respectively. 48 hours after transduction, each medium of the host cell including the virus was filtered using a filter with a size of 0.45 pm to remove cell debris, thereby obtaining viruses.

CAFs and normal fibroblasts were cultured in a 6-well plate, respectively. When their growth reached 90% or more, polybrene was used to induce infection of CAFs and normal fibroblasts with the lentiviruses obtained above, respectively. Each cell was infected with the lentivirus twice, and adherent-cultured for about 14 days while replacing media every about 48 hours. The CAFs and fibroblasts were induced into iPCs by acquiring pluripotency. At this time, adherent-culture was performed using a plate coated with a protein geltrex.

The gene combinations transduced into the CAFs and fibroblasts via the lentivirus are as follows.
Oct4
Sox2
Oct4/Sox2
miR125b
Sox2/miR125b

A composition of the used medium is as follows:

DMEM/F12+10% knockout serum replacement(KSR) +1% NEAA +1 mM L-glutamine +1% penicillin streptomycin (P/S) +0.1 mM β-mercaptoethanol +10 ng/ml basic fibroblast growth factor (bFGF) +30 ng/ml insulin-like growth factor 2 (IGFII)

1.3. Differentiation of Hematopoietic Stem Cells from iPCs

To prepare hematopoietic stem cells from the obtained iPCs, when colonies were observed by the culturing, the adherent cell culturing was changed to suspension cell culturing in a poly-hema-coated plate. When the culturing method is changed to the suspension cell culturing, cells may grow while forming a spheroid. The cells were continuously cultured for about 14 days while replacing the medium every about 48 hours. In detail, differentiation of hematopoietic stem cells was induced by adding the medium for the first about 7 days and changing the medium every other day for the remaining period. A composition of the used medium is as follows:

KnockOut DMEM (KO-DMEM) +20% fetal calf serum (FCS) +1% penicillin streptomycin (P/S) +1% NEAA +1 mM L-glutamine +0.1 mM β-mercaptoethanol

1.4. Differentiation of Macrophages from Hematopoietic Stem Cells

To induce differentiation of macrophages from the differentiated hematopoietic stem cells, spherical hematopoietic stem cells obtained by the culturing were separated into single cells using accutase, followed by adherent-cell culture. To induce differentiation into macrophages, the cells were cultured for about 1 week while replacing the medium every other day. A composition of the used medium is as follows:

RPM11640 +10% FBS +1% (P/S) +10 μg/ml IL-4 +10 μg/ml M-CSF

### EXPERIMENTAL EXAMPLE 1

Identification of iPCs Prepared from CAFs

To examine cell morphology and protein changes by gene transduction in the iPCs obtained in Example 1.2, fluorescence microscopy was utilized. In detail, the expression level of Sox2, Oct4, and miR125b were detected using mCitrin fluorescent protein, GFP fluorescent protein, and mCherry fluorescent protein, which are attached to Sox2, Oct4 and miR125b, respectively. To observe cell morphology, cells were imaged using a Nikon Eclipse Ts2P fluorescence microscope. GFP fluorescence was detected using a 39002 filter set (Chroma) after excitation at a wavelength of 470 nm, and mCitrin and mCherry were detected using a 39004 filter set (Chroma) after excitation at a wavelength of 525 nm.

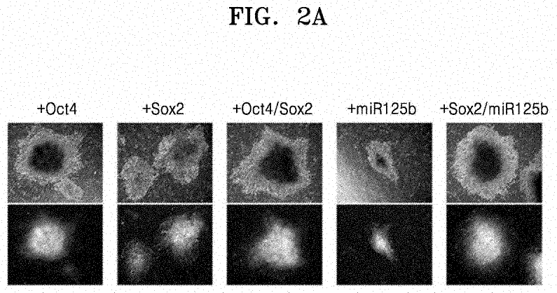

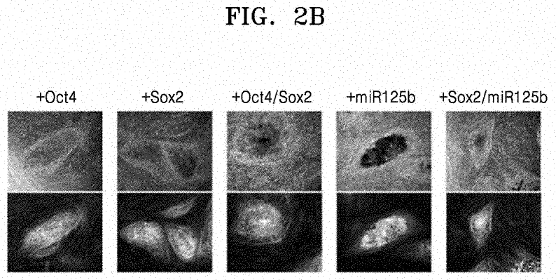

FIG. 2A shows results of observing iPCs under a fluorescence microscope, the iPCs obtained by transducing Oct4, Sox2, Oct4/Sox2, miR125b, or Sox2/miR125b into CAFs.

FIG. 2B shows results of observing iPCs under a fluorescence microscope, the iPCs obtained by transducing Oct4, Sox2, Oct4/Sox2, miR125b, or Sox2/miR125b into normal fibroblasts.

As shown in FIGS. 2A and 2B, the iPCs prepared by overexpression of Oct4, Sox2 and/or miR125b transduced into CAFs and normal fibroblasts were able to form stem cell colonies having the same morphology as embryonic stem cells. Further, the expression of fluorescent proteins according to the each transduced gene was observed.

Therefore, we could confirm that Oct4, Sox2 and/or miR125b transduced into CAFs and normal fibroblasts were overexpressed, and as a result, the prepared cells could form stem cell colonies, indicating successful differentiation into iPCs.

Further, to examine whether expression of Oct4, Sox2, and Nanog which are pluripotency markers was increased by single or combined introduction of Oct4, Sox2, and/or miR125b, qRT-PCR was performed.

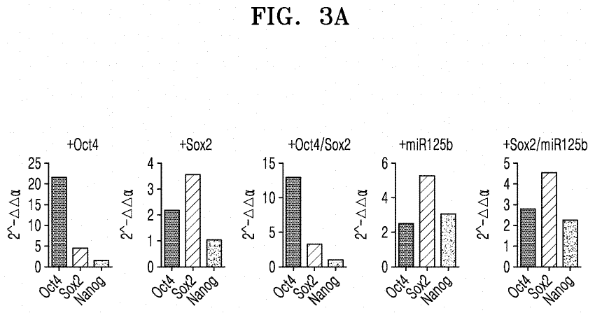

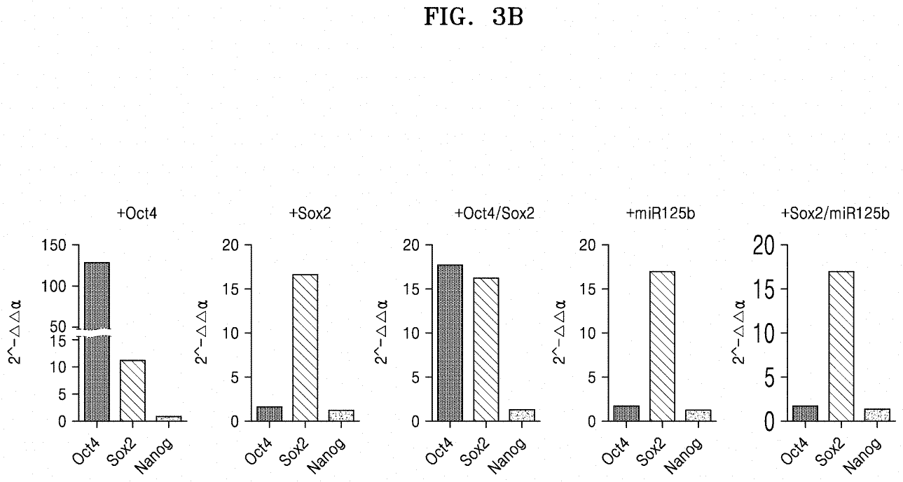

FIG. 3A shows qRT-PCR results of analyzing the expression of Oct4, Sox2, and Nanog, which are pluripotency markers, in iPCs, the iPCs being obtained by transducing Oct4, Sox2, Oct4/Sox2, miR125b, or Sox2/miR125b into CAFs.

FIG. 3B shows qRT-PCR results of analyzing the expression of Oct4, Sox2, and Nanog, which are pluripotency markers, in iPCs, the iPCs being obtained by transducing Oct4, Sox2, Oct4/Sox2, miR125b, or Sox2/miR125b into normal fibroblasts.

As shown in FIGS. 3A and 3B, when Oct4 or both Oct4 and Sox2 was/were overexpressed in CAFs, Oct4, Sox2, and Nanog showed similar expression levels with each other. In contrast, even though only Oct4 was overexpressed in normal fibroblasts, the degree of Sox2 expression level was high enough to the case when Sox2 was overexpressed. These results support the previous experimental results reporting that only Oct 4 expression is sufficient to differentiate normal fibroblasts into hematopoietic stem cells. Meanwhile, when Sox2, miR125b, or Sox2/miR125b was overexpressed in CAFs, Oct4, Sox2, and Nanog showed similar expression levels with each other. In normal fibroblasts, all the three genes also showed similar results. Further, when miR125b was overexpressed in CAFs, Nanog showed a relatively high expression.

Therefore, it was confirmed that both CAF and normal fibroblast could be successfully differentiated into the iPCs by the method stated above, and when the iPCs were differentiated from CAFs and normal fibroblasts even by the same differentiation method, they showed different cell characteristics from each other.

### EXPERIMENTAL EXAMPLE 2

Identification of Hematopoietic Stem Cells

To examine whether the hematopoietic stem cells obtained in Example 1.3 had differentiation ability, embryonic body (EB) formation which is a characteristic of stem cells was examined. In detail, colonies formed by gene transduction were separated into single cells, and then cultured in a poly-hema-coated plate, followed by observation under a microscope daily.

Cells acquiring stem cell characteristics show three-dimensional spherical cell growth in a non-adherent state, whereas cells having no stem cell characteristics do not show three-dimensional spherical cell growth.

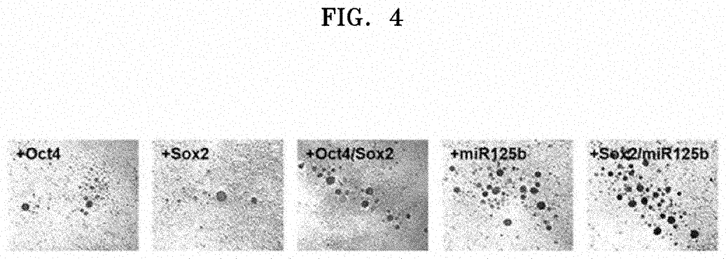

FIG. 4 shows results of analyzing whether iPCs obtained by transduction of Oct4, Sox2, Oct4/Sox2, miR125b, or Sox2/miR125b into CAFs have EB-forming ability.

As shown in FIG. 4, when Oct4, Sox2, Oct4/Sox2, miR125b, or Sox2/miR125b was transduced, all the iPCs obtained thereby showed three-dimensional spherical cell growth in a non-adherent state, indicating that they had EB-forming ability. Further, differentiation of hematopoietic stem cells from EB of the iPCs prepared by the method was induced.

As a result, it was confirmed that iPCs prepared by the method had pluripotent stem cell characteristics.

Further, to examine differentiation ability of the iPCs into hematopoietic stem cells and blood cells, flow cytometry was performed to examine expression of CD34 protein which is a hematopoietic stem cell membrane marker. First, cells were separated into single cells by treatment with Accutase (gibco), and then washed and blocked with a 1% FBS/PBS solution. Then, APC-conjugated antibody was incubated for CD34 detection. To examine expression of CD34 protein, APC Mouse Anti-Human CD34 available from BD was used, and as a control group, APC Mouse IgG1-Isotype was used to perform flow cytometry. Data were analyzed using a Flowjo program.

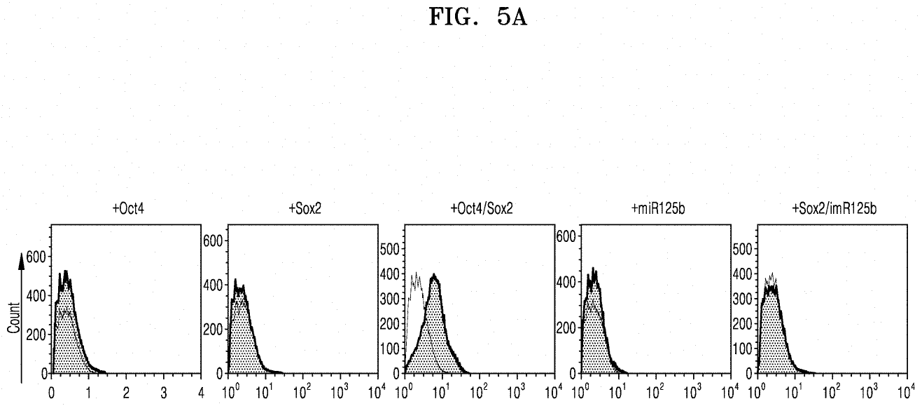

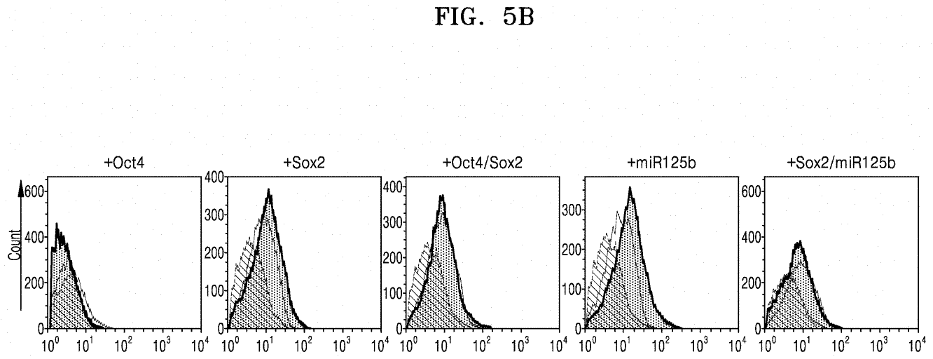

FIG. 5A shows flow cytometry results of analyzing the expression of CD34 which is a membrane protein of hematopoietic stem cells differentiated from iPCs which were obtained by transducing Oct4, Sox2, Oct4/Sox2, miR125b, or Sox2/miR125b into CAFs.

FIG. 5B shows flow cytometry results of analyzing the expression of CD34 which is a membrane protein of hematopoietic stem cells differentiated from iPCs which were obtained by transducing Oct4, Sox2, Oct4/Sox2, miR125b, or Sox2/miR125b into normal fibroblasts.

As shown in FIGS. 5A and 5B, when hematopoietic stem cells derived from CAFs were treated with Oct4 and Sox2 at the same time (Oct4/Sox2), the highest CD34 expression was observed. When normal fibroblasts were treated with Sox2 or miR125b alone, or both Oct4 and Sox2, all showed the similar results.

Further, to examine whether the cells had ability to differentiate into a mesoderm, expression of GATA2 and Brachy which are mesodermal lineage markers was examined by qRT-PCR.

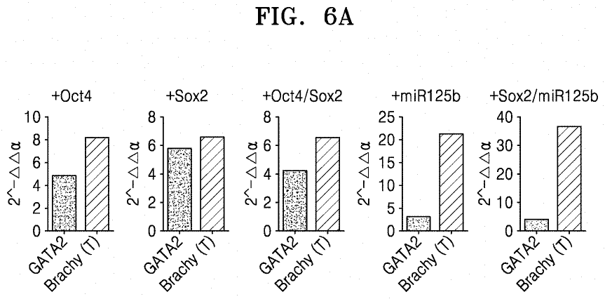

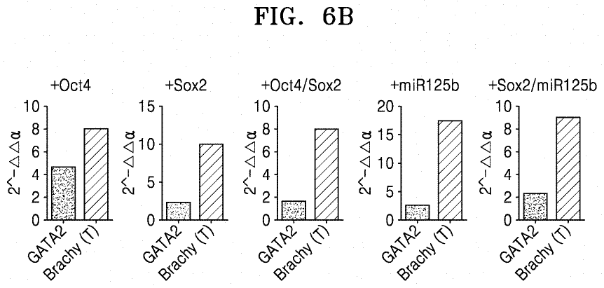

FIG. 6A shows qRT-PCR results of analyzing the expression of GATA2 and Brachy in hematopoietic stem cells differentiated from the iPCs which were obtained by transducing Oct4, Sox2, Oct4/Sox2, miR125b, or Sox2/miR125b into CAFs.

FIG. 6B shows qRT-PCR results of analyzing the expression of GATA2 and Brachy in hematopoietic stem cells differentiated from the iPCs which were obtained by transducing Oct4, Sox2, Oct4/Sox2, miR125b, or Sox2/miR125b into normal fibroblasts.

As shown in FIGS. 6A and 6B, the hematopoietic stem cells differentiated from CAFs showed a significant increase of Brachy (T) in the presence of miR125b, whereas the hematopoietic stem cells differentiated from normal fibroblasts showed no significant change.

### EXPERIMENTAL EXAMPLE 3

Identification of Macrophages

Further, to examine whether the obtained hematopoietic stem cells had ability to differentiate into macrophages by Example 1.4, the formed EB was separated into single cells, which were then cultured. After culturing with a macrophage colony-stimulating factor (M-CSF) and cytokines for 7 days, expression of CD45 which is a cell membrane marker for blood cells/monocytes was analyzed by flow cytometry using an antigen-antibody reaction.

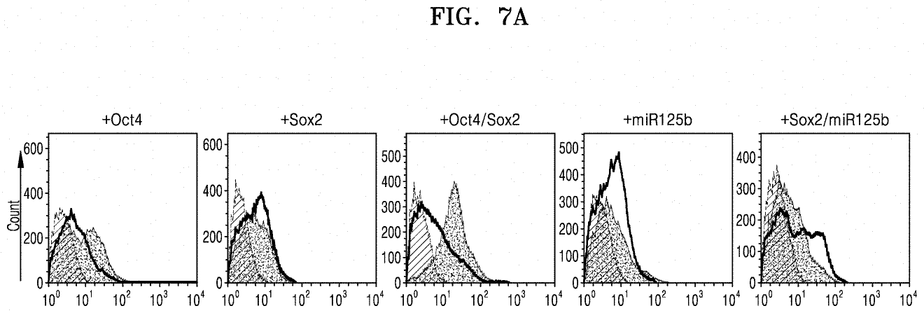

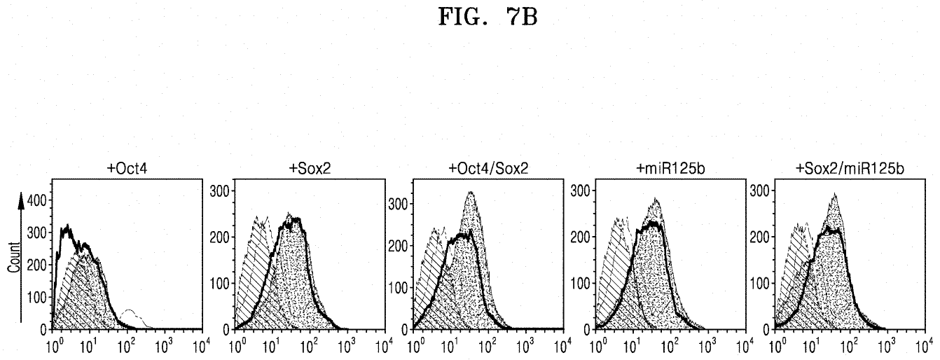

FIG. 7A shows flow cytometry results of analyzing the expression of CD45 which is a membrane protein in blood cells/monocytes which were obtained by transducing Oct4, Sox2, Oct4/Sox2, miR125b, or Sox2/miR125b into CAFs.

FIG. 7B shows flow cytometry results of analyzing the expression of CD45 which is a membrane protein in blood cells/monocytes which were obtained by transducing Oct4, Sox2, Oct4/Sox2, miR125b, or Sox2/miR125b into normal fibroblasts.

As shown in FIGS. 7A and 7B, when the blood cells/monocytes cells derived from CAFs were treated with Oct4 and Sox2 at the same time, the highest CD45 expression was observed.

Further, to examine whether the obtained blood cells/monocytes cells had ability to differentiate into macrophages, changes in C/EBPα, PU.1, MXIL1 and GATA1 expression in the blood cells/monocytes cells were examined by qRT-PCR. It is known that C/EBPα is a critical factor for differentiation/development of blood cells, and PU.1 is an inducer of differentiation/development of monocyte/macrophage. Further, MXIL1 and GATA1 are factors that contribute to differentiation of mesodermal lineages and blood cells/monocytes. These genes are reported to play a role in differentiating cells from hematopoietic stem cells/stem-cells to determine characteristics of blood cells and to develop their function.

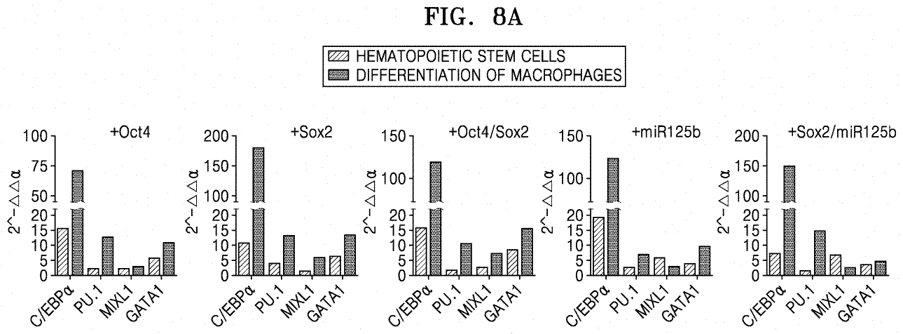

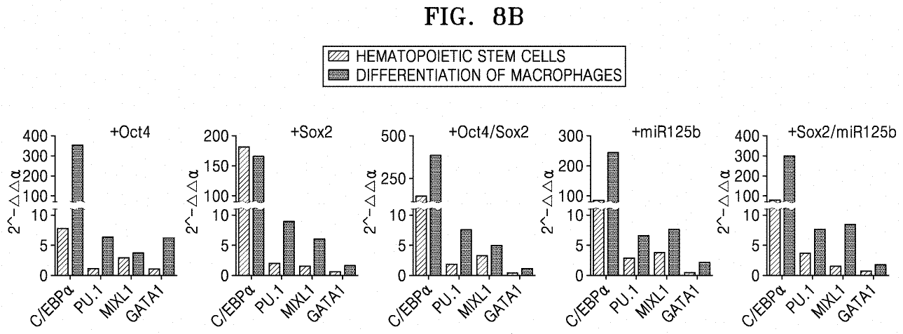

FIG. 8A shows qRT-PCR results of analyzing the expression of C/EBPα, PU.1, MXIL1, and GATA1 in macrophageswhich were obtained by transducing Oct4, Sox2, Oct4/Sox2, miR125b, or Sox2/miR125b into CAFs.

FIG. 8B shows qRT-PCR results of analyzing the expression of C/EBPα, PU.1, MXIL1, and GATA1 in macrophageswhich were obtained by transducing Oct4, Sox2, Oct4/Sox2, miR125b, or Sox2/miR125b into normal fibroblasts.

As shown in FIGS. 8A and 8B, expression of C/EBPα which is a critical factor for differentiation/development of macrophages was greatly increased, and expression of PU.1 which is an inducer of differentiation/development of monocytes was also increased, after differentiation of macrophages. Further, expression of MXIL1 and GATA1 which contribute to differentiation of mesodermal lineages and monocytes was increased, when simultaneous expression of Oct4 and Sox2 was induced, as compared with single introduction of Oct4 or Sox2. The macrophages derived from normal fibroblasts showed increased CD45 expression, when Sox2 or miR125b was treated alone or co-treated with Oct4, rather than Oct4 alone. C/EBPα expression was increased by Sox2 introduction, and the highest expression thereof was observed when induced together with Oct4. Further, expression of PU.1 and MXIL1 which contribute to differentiation/development of monocytes was increased in the macrophages derived from normal fibroblasts by Sox2 introduction.

### EXPERIMENTAL EXAMPLE 4

Evaluation of Functionality of Macrophage

To examine whether the macrophages obtained in Example 1.4 had functionality, their phagocytic function was evaluated. In detail, 1 μm-sized latex beads were used. Differentiation of macrophages from hematopoietic stem cells was induced for 1 week, and then 1 μm-sized latex beads (sigma) stabilized in a cell culture medium were added to cells, and allowed to react for 90 minutes. The cells were washed with cold phosphate-buffered saline (PBS), and then fixed in a 4% paraformaldehyde solution. Cell morphology and phagocytosis were observed under a microscope.

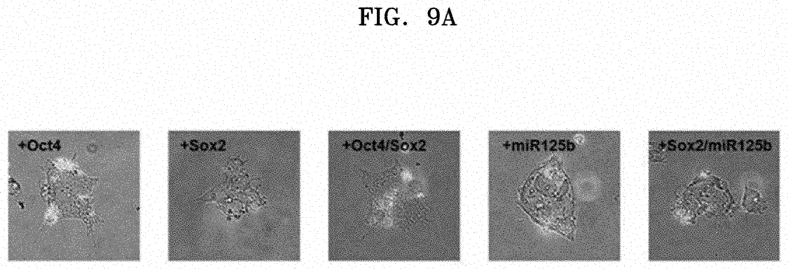

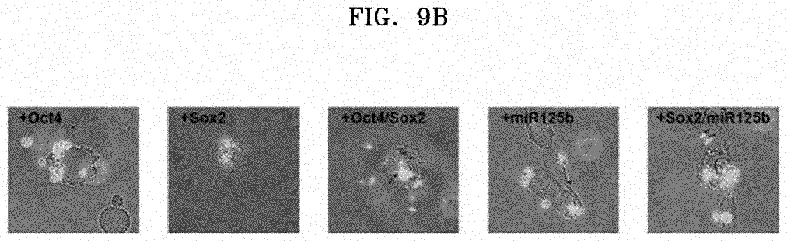

FIG. 9A shows results of evaluating phagocytic function of macrophages which were obtained by transducing Oct4, Sox2, Oct4/Sox2, miR125b, or Sox2/miR125b into CAFs.

FIG. 9B shows results of evaluating phagocytic function of macrophages which were obtained by transducing Oct4, Sox2, Oct4/Sox2, miR125b, or Sox2/miR125b into normal fibroblasts.

As shown in FIGS. 9A and 9B, when the macrophages derived from CAFs or normal fibroblasts were co-cultured with latex beads, intracellular uptake of the latex beads was observed. Further, the cell morphology changed similarly to that of macrophage.

Therefore, it was confirmed that the macrophages derived from CAFs or normal fibroblasts by the method according to an aspect were functional macrophages having the phagocytic function.

According to a method of reprogramming CAFs according to an aspect, macrophages may be prepared with a high yield in a short period of time, and the tumor microenvironment may be suppressed and macrophages reprogrammed from CAFs may be capable of eliminating cancer cells. Therefore, the macrophages may be usefully applied as an anticancer agent or an anticancer adjuvant.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A method of differentiating CAFs (cancer-associated fibroblasts) into macrophages, the method comprising:
enhancing expression of Oct4 and Sox2 in CAFs and culturing the CAFs in a medium to differentiate the CAFs into iPCs (induced pluripotent stem cells);
culturing the iPCs in a medium to differentiate the iPCs into hematopoietic stem cells; and
culturing the hematopoietic stem cells in a medium to differentiate the hematopoietic stem cells into macrophages.

2. The method of claim 1, wherein, in the differentiation into iPCs, expression of miR125b is further enhanced in the CAFs.

3. The method of claim 1, wherein, in the differentiation into iPCs, the CAFs are cultured in a medium comprising a serum replacement, β-mercaptoethanol, basic fibroblast growth factor (bFGF), or a combination thereof.

4. The method of claim 1, wherein, in the differentiation into iPCs, the culturing is adherent-culturing.

5. The method of claim 1, wherein, in the differentiation into iPCs, the culturing is performed for 10 days to 20 days.

6. The method of claim 1, wherein, in the differentiation into hematopoietic stem cells, the iPCs are cultured in a medium comprising β-mercaptoethanol, fetal calf serum (FCS), or a combination thereof.

7. The method of claim 1, wherein, in the differentiation into hematopoietic stem cells, the culturing is suspension culturing.

8. The method of claim 1, wherein, in the differentiation into hematopoietic stem cells, the culturing is performed for 10 days to 20 days.

9. The method of claim 1, wherein, in the differentiation into macrophages, the hematopoietic stem cells are cultured in a medium comprising IL-4, M-CFS, or a combination thereof.

10. The method of claim 1, wherein, in the differentiation into macrophages, the culturing is adherent-culturing.

11. The method of claim 1, wherein, in the differentiation into macrophages, the culturing is performed for 5 days to 10 days.

12. The method of claim 3, wherein a concentration of the serum replacement is 1% by weight to 20% by weight, a concentration of the 3-mercaptoethanol is 0.05 mM to 1.5 mM, and a concentration of the bFGF is 1 ng/ml to 20 ng/ml in the total medium.

13. The method of claim 6, wherein a concentration of the 3-mercaptoethanol is 0.05 mM to 1.5 mM, and a concentration of the FCS is 10% by weight to 30% by weight of the total medium.

14. The method of claim 9, wherein a concentration of the IL-4 is 1 μg/ml to 20 μg/ml, and a concentration of the M-CFS is 1 μg/ml to 20 μg/ml.

15. Macrophages prepared by the method of claim 1.

16. A pharmaceutical composition for preventing or treating cancer, the pharmaceutical composition comprising macrophages prepared by the method of claim 1.
